(19) United States
(12) Patent Application Publication
(10) Pub. No.: US 2008/0014256 A1
(43) Pub. Date: Jan. 17, 2008

(54) Oral formulations comprising tigecycline

(75) Inventors: Christopher R. Diorio, Campbell Hall, NY (US); Syed M. Shah, East Hanover, NJ (US); Kadum A. Ali, Congers, NY (US)

Correspondence Address: WYETH/FINNEGAN HENDERSON, LLP, 901 NEW YORK AVENUE, NW, WASHINGTON, DC, 20001-4413, US

(73) Assignee: Wyeth

(21) Appl. No.: 11/642,509

(22) Filed: Dec. 21, 2006

Related U.S. Application Data
(60) Provisional application No. 60/753,035, filed on Dec. 22, 2005.

(51) Int. Cl. A61K 31/65; A61K 9/20; A61K 9/48; A61P 31/04

(52) U.S. Cl. 424/452; 424/465; 514/152

(57) ABSTRACT

Disclosed herein are pharmaceutical compositions comprising tigecycline for oral administration. The composition can comprise tigecycline having at least one enteric coating.

### CROSS-REFERENCE TO RELATED APPLICATIONS

This application claims the benefit of U.S. Provisional Application Ser. No. 60/753,035, filed Dec. 22, 2005, which is incorporated herein by reference in its entirety.

In one embodiment, this invention relates to oral formulations comprising tigecycline.

Tigecycline is a glycylcycline antibiotic, i.e., a t-butylglycyl substituted naphthacenecarboxamide free base, and an analog of the semisynthetic tetracycline, minocycline.

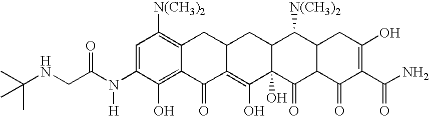

Tetracyclines such as chlortetracycline hydrochloride (Aureomycin) and oxytetracycline (Terramycin) are safe and have been used therapeutically as broad-spectrum antibiotics since 1948. However, the emergence of resistance to these antibiotics had limited their continued widespread usage. Tigecycline was thus developed as an agent to potentially restore therapeutic utility to tetracyclines by overcoming tetracycline resistance mechanisms. Tigecycline may also provide activity against emerging multi-drug resistant pathogens. Glycylcyclines, including tigecycline, are active against many antibiotic-resistant gram-positive pathogenic bacteria, such as methicillin-resistant Staphylococcus aureus, penicillin-resistant Streptococcus pneumoniae, and vancomycin-resistant enterococci (Weiss et al., 1995; Fraise et al., 1995). Tigecycline is also active against bacterial strains carrying the two major forms of tetracycline resistance, efflux and ribosomal protection (Schnappinger and Hillen, 1995).

Minocycline is currently available in oral and IV forms. Although an intravenous formulation of tigecycline has been prepared, simple oral immediate release prototypes containing tigecycline have resulted in poor bioavailability in animals. (Petersen et al., Antimicrobial Agents and Chemotherapy, April 1999, Vol. 43, No. 4 p. 738-744.)

Tigecycline is very soluble in water with solubility greater than 295 mg/mL over the entire pH range of 1 to 14. However, cell monolayer permeability studies of tigecycline (1 mM in ethanol and buffer, pH 6 to 6.4) show a low value of 0.4 nm s⁻¹, suggesting a low GI permeability, which is consistent with the low oral bioavailability found in animals.

Accordingly, there remains a need to develop an oral formulation of tigecycline.

### BRIEF DESCRIPTION OF THE DRAWINGS

FIG. 1 is a plot of percent release of tigecycline (y-axis) versus time x-axis, min);

FIG. 2 shows the analytical performance of tigecycline in monkey plasma, low QC (quality control)-300 ng/mL as a plot of tigecycline plasma concentration (y-axis) vs. curve number x-axis);

FIG. 3 shows the analytical performance of tigecycline in monkey plasma, mid QC A-663 ng/mL as a plot of tigecycline plasma concentration (y-axis) vs. curve number x-axis);

FIG. 4 shows the analytical performance of tigecycline in monkey plasma, mid QC B-556 ng/mL as a plot of tigecycline plasma concentration (y-axis) vs. curve number x-axis);

FIG. 5 shows the analytical performance of tigecycline in monkey plasma, high QC-3000 ng/mL as a plot of tigecycline plasma concentration (y-axis) vs. curve number x-axis);

FIG. 6 is a plot of plasma concentration (y-axis) vs. time (x-axis) profile of tigecycline in monkeys after a single intravenous dose of 5 mg/kg;

FIG. 7 is a plot of tigecycline plasma concentration (y-axis) vs. curve number x-axis), showing the analytical performance of tigecycline assay in monkey plasma: low QC (quality control)−30 ng/mL;

FIG. 8 is a plot of tigecycline plasma concentration (y-axis) vs. curve number x-axis), showing the analytical performance of tigecycline assay in monkey plasma: middle QC−300 ng/mL;

FIG. 9 is a plot of tigecycline plasma concentration (y-axis) vs. curve number x-axis), showing the analytical performance of tigecycline assay in monkey plasma: high QC−800 ng/mL; and

FIG. 10 is a plot of plasma concentration of tigecycline (ng/ml, y-axis) vs. time (h, x-axis) after a single oral dose (100 mg encapsulated microparticulate capsule) in fasted male cynomolgus monkey.

One embodiment of the present invention provides a pharmaceutical composition comprising tigecycline having at least one enteric coating. In one embodiment, the composition is in oral dosage form. The enteric coated tigecycline compositions may further comprise one or more of the further ingredients described herein.

In one embodiment, “having an enteric coating” refers to surrounding a bulk of tigecycline. In another embodiment, the enteric coating surrounds substantially each Tigecycline particle. “Coating” can comprise either a coating or subcoating. “Coating,” or “surrounds” as used herein, may range, for example, from at least partially coating or surrounding up to and including a complete coating or surrounding. In one embodiment, coating or surrounding refers to substantially coating, such as 90%, 95%, and 99% coating by weight. In one embodiment, the enteric coating may be sufficiently uniform to confer physical stability to the tigecycline, e.g., by preventing degradation by any method disclosed herein.

In one embodiment, an “enteric coating” can allow at least a substantial portion of a formulation to pass through the stomach and disintegrate in the intestines. Exemplary materials for the preparation of enteric coatings include, but are not limited to, hydroxypropylmethylcellulose, hydroxyethylcellulose, methylhydroxyethylcellulose, sodium carboxymethylcellulose, hydroxypropylcellulose, polyvinyl pyrrolidone, dimethylaminoethyl methacrylatemethylacrylate acid ester copolymer, anionic acrylic resins such as methacrylic acid/methyl acrylate copolymer and methacrylic acid/ethyl acrylate copolymer, ethylacrylate-methylmethacrylate copolymer, hydroxypropylmethylcellulose acetate succinate (HPMCAS), hydroxypropylmethylcellulose phthalate (HPMCP), cellulose acetate phthalate (CAP), carboxymethylcellulose acetate phthalate (CMCAP), shellac, methylcellulose, and ethylcellulose, and blends and copolymers thereof.

In one embodiment, the enteric coating may be formed by methods known in the art for forming polymeric films.

In one embodiment, the composition further comprises a seal coat. In one embodiment, the seal coat is positioned underneath the enteric coat. In another embodiment, the composition can contain at least one additional seal coat that overcoats the enteric coat, which in turn overcoats a first seal coat. In one embodiment, the seal coat comprises any material suitable for forming enteric coatings, such as hydroxypropyl cellulose, polyvinyl pyrrolidone, sodium carboxymethylcellulose, and hypromellose, or any other enteric coating material disclosed herein.

In one embodiment, the at least one enteric coating can protect tigecycline from substantial degradation. Tigecycline may have at least two degradation mechanisms. At low pH, epimerization of the dimethylamino group at 4-position has been identified as a major degradation route. At pH higher than 7.4, the degradation mechanism shifts to oxidation, as the phenolic groups can become deprotonated. Tigecycline can, for example, be stabilized in both solid and solution states by eliminating oxygen. Once oxygen is eliminated, the pH of optimum stability shifts from 4.5 to 8 where epimerization is at its minimum.

In one embodiment, the enteric coating allows delivery of the oral formulation to the gastrointestinal (GI) tract for selective release into the gastrointestinal tract. such as the lower gastrointestinal tract. The gastrointestinal tract includes the upper and lower GI tract. The upper GI tract includes the stomach and esophagus. In one embodiment, “lower gastrointestinal tract” as used herein refers to the ileum and large intestine. “Ileum” as used herein refers to a third part of the small intestine that continues to the duodenum and jejunum. “Large intestine” as used herein comprises the cecum, colon, and rectum. “Cecum” refers to a blind sack (cul-de-sac) starting from the large intestine and in one end of which the ileum opens.

In one embodiment, the oral formulation does not release a substantial amount of tigecycline in the stomach and a substantial release occurs when the formulation reaches the gastrointestinal tract, such as the lower GI tract.

In one embodiment, the composition further comprises at least one chelating agent. Calcium binds to tetracyclines, which reduce its water solubility. There may be a 30 to 40% loss of tigecycline due to precipitation of the calcium complex at pH 7.4. Thus, calcium binding and subsequent precipitation of the calcium/tigecycline salt may be at least partially responsible for low oral bioavailability. Exemplary chelating agents include ethylenediaminetetraacetic acid (EDTA), O,O′-bis(2-aminoethyl)ethyleneglycol-N,N,N′,N′-tetraacetic acid (EGTA), citrates, and tartrates.

In one embodiment, the composition further comprises at least one base. In one embodiment, the at least one base provides the composition with a microenvironment having a pH ranging from 4 to 8.5 when released, such as a pH ranging from 7.8 to 8.5 when released. In one embodiment, the pH of the microenvironment refers to the pH of the area immediately surrounding the composition. In another embodiment, the microenvironment refers to the area inside the seal coat. Exemplary bases include, but are not limited to, phosphates, such as at least one sodium phosphate, carbonates such as sodium and potassium carbonate, bicarbonates, such as sodium and potassium bicarbonate, citrates, such as sodium citrate, and tartrates.

Additionally, in some embodiments, buffer species can negatively affect the stability of tigecycline. In one embodiment, the at least one base may be capable of countering the effects of such buffer species.

In one embodiment, the composition further comprises at least one biopolymer. For example, in embodiments where the composition is used to treat infections in the GI tract, such as the inner or lower GI tract, the at least one biopolymer can act as an adhesive to the inner GI tract and therefore allow for enhanced absorption of tigecycline. Exemplary biopolymers include, but are not limited to, hypromellose and xanthan gum, and carbomer.

“Pharmaceutical composition” as used herein refers to a medicinal composition. The pharmaceutical composition may contain at least one pharmaceutically acceptable carrier.

In one embodiment, the composition further comprises at least one inert, pharmaceutically-acceptable excipient or carrier. “Pharmaceutically acceptable excipient” as used herein refers to pharmaceutical carriers or vehicles suitable for administration of tigecycline including any such carriers known to those skilled in the art to be suitable for oral administration.

Suitable excipients include, for example, (a) fillers or extenders such as starches, lactose, sucrose, glucose, mannitol, and silicic acid; (b) binders such as cellulose and cellulose derivatives (such as hydroxypropylmethylcellulose, hydroxypropylcellulose, and carboxymethylcellulose), alginates, gelatin, polyvinylpyrrolidone, sucrose, and acacia; (c) humectants such as glycerol; (d) disintegrating agents such as sodium starch glycolate, croscarmellose, agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents, such as cetyl alcohol and glycerol monostearate, fatty acid esters of sorbitan, poloxamers, and polyethylene glycols; (h) absorbents such as kaolin and bentonite clay; (i) lubricants such as talc, calcium stearate, magnesium stearate, solid polyethylene glycols, sodium lauryl sulfate, and mixtures thereof; and (j) glidants (antiadherents) such as talc, and silicone dioxide. Other suitable excipients include, for example, sodium citrate or dicalcium phosphate. The dosage forms may also comprise buffering agents.

Oral formulations may also employ fillers in soft and hard-filled gelatin capsules using such excipients as lactose or milk sugar as well as high molecular weight polyethylene glycols.

The pharmaceutical compositions may optionally contain opacifying agents and colorants. They may also be in a form capable of controlled or sustained release. Examples of embedding compositions that can be used for such purposes include polymeric substances and waxes.

Where the composition is a suspension containing powdered tigecycline, the suspension can further comprise, for example, from about 0.05 to 5% of suspending agent by weight, syrups containing, for example, from about 10 to 50% of sugar by weight, and elixirs containing, for example, from about 20 to 50% ethanol by weight.

The pharmaceutical compositions disclosed herein may contain, for example, an amount ranging from about 25 to about 90% of the active ingredient by weight relative to the total weight of the composition, or from about 5% and 60% by weight.

The tigecycline can be provided as a pharmaceutically acceptable salt. The terms “pharmaceutically acceptable salt” can refer to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, mucbnic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Pharmaceutically acceptable salts may be derived from amino acids, including but not limited to cysteine. Other acceptable salts may be found, for example, in Stahl et al., Pharmaceutical Salts Properties, Selection, and Use, Wiley-VCH; 1 st edition (Jun. 15, 2002).

Another embodiment provides a method of preparing a pharmaceutical composition comprising coating a tigecycline with at least one enteric coating. The coating can be performed using any known process in the art, such as by introducing the tigecycline into a fluid bed processor (or other coating device, such as a pan coater) containing the enteric coating material. Prior to its introduction into the coating device, the tigecycline can be combined with one or more of at least one base/buffer, at least one chelating agent, at least one biopolymer, and other ingredients suitable for the oral formulation.

Another embodiment provides a method of treating at least one bacterial infection, comprising:

orally administering to a subject in need thereof a pharmaceutical composition comprising a therapeutically effective amount of tigecycline having at least one enteric coating.

Another embodiment provides a method of treating antibiotic associated pseudomembranous colitis caused by C. difficile and enterocolitis caused by S. aureus and associated methicillin resistant strains comprising:

orally administering to a subject in need thereof a pharmaceutical composition comprising a therapeutically effective amount of tigecycline having at least one enteric coating.

In one embodiment, “therapeutically effective amount” refers to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined by one of ordinary skill in the art. The selected dosage level can depend upon the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved.

In one embodiment, the subject treated can be a mammal, such as a human. In one embodiment, the subject is suspected of having a bacterial infection, e.g., shows at least one symptom associated with the infection. In another embodiment, the subject is one susceptible to having the bacterial infection, for example, a subject genetically disposed to having the disease.

“Treating” as used herein refers to both therapeutic treatment and prophylactic/preventative measures. Those in need of treatment may include individuals already having a particular medical disease as well as those at risk for the disease (i.e., those who are likely to ultimately acquire the disorder). A therapeutic method results in the prevention or amelioration of symptoms or an otherwise desired biological outcome and may be evaluated by improved clinical signs, delayed onset of disease, reduced/elevated levels of lymphocytes and/or antibodies, etc.

In one embodiment, the administering is performed with a nasal-gastric tube.

Actual dosage levels of tigecycline in the pharmaceutical compositions of this invention may be varied so as to obtain the therapeutically effective amount necessary to achieve the desired therapeutic response for a particular patient.

Generally dosage levels of about 0.1 μg/kg to about 50 mg/kg, such as a level ranging from about 5 to about 20 mg of active compound per kilogram of body weight per day, can be administered topically, orally or intravenously to a mammalian patient. Other dosage levels range from about 1 μg/kg to about 20 mg/kg, from about 1 μg/kg to about 10 mg/kg, from about 1 μg/kg to about 1 mg/kg, from 10 μg/kg to 1 mg/kg, from 10 μg/kg to 100 μg/kg, from 100 μg to 1 mg/kg, and from about 500 μg/kg to about 5 mg/kg per day. If desired, the effective daily dose may be divided into multiple doses for purposes of administration, e.g., two to four separate doses per day.

In one embodiment, the pharmaceutical composition can be administered once or twice per day.

In one embodiment, the tigecycline is multi-particulate. As used herein, “multi-particulate tigecycline” refers to a collection of tigecycline particles. In one embodiment, the multi-particulate tigecycline has a mean particle size ranging from 0.3 mm to 1.5 mm. The multi-particulate tigecycline can be provided as a powder, or provided as a capsule encased within a shell, or any other dosage form as described herein.

In one embodiment, dosage forms for oral administration include, but are not limited to, capsules, tablets, pills, powders (e.g., dispersible powders, suspensions containing such powders), dragees, granules, and lyophilized cakes and powders. Such forms may include forms that dissolve or disintegrate quickly in the oral environment. In another embodiment, the oral dosage form slows the dissolution of the drug immediately following oral administration and allows a substantial portion of the dissolution to occur in the GI tract, such as the lower GI tract. In one embodiment, the dosage form (e.g., powders, cakes) is provided in vials or other suitable containers.

In one embodiment, the pharmaceutical composition comprises a compressed tablet containing tigecycline in an amount ranging from 100 mg to 300 mg.

In one embodiment, the pharmaceutical composition comprises enteric coated multi-particulate pellets incorporated into a hard gelatin capsule, and each pellet comprising tigecycline and microcrystalline cellulose, and a combination of one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises an enteric coated tablet comprising tigecycline and microcrystalline cellulose, and further comprises one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises multi-particulate pellets incorporated into an enteric coated soft gelatin capsule, and each pellet comprising tigecycline and microcrystalline cellulose, and one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises an enteric coated soft liquid gel capsule, and further comprising a non-aqueous solution of tigecycline, and one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises a capsule or bi-layer tablet comprising both an immediate release portion and an extended release portion. In one embodiment, “extended release” involves release of substantially all of the tigecycline over a time period of at least 4 hours, such as a time period of at least 6 hours, at least 12 hours, at least 24 hours, or at least 48 hours.

In one embodiment, the pharmaceutical composition may be used as a treatment against drug-resistant bacteria. For example, it may be active against methicillin-resistant Staphylococcus aureus, penicillin-resistant Streptococcus pneumoniae, vancomycin-resistant enterococci (D. J. Beidenbach et. al., Diagnostic Microbiology and Infectious Disease 40:173-177 (2001); H. W. Boucher et. al., Antimicrobial Agents & Chemotherapy 44:2225-2229 (2000); P. A. Bradford Clin. Microbiol. Newslett. 26:163-168 (2004); D. Milatovic et. al., Antimicrob. Agents Chemother. 47:400-404 (2003); R. Patel et. al., Diagnostic Microbiology and Infectious Disease 38:177-179 (2000); P. J. Petersen et. al., Antimicrob. Agents Chemother. 46:2595-2601 (2002); and P. J. Petersen et. al., Antimicrob. Agents Chemother. 43:738-744(1999), and against organisms carrying either of the two major forms of tetracycline resistance: efflux and ribosomal protection (C. Betriu et. al., Antimicrob. Agents Chemother. 48:323-325 (2004); T. Hirata et. al. Antimicrob. Agents Chemother. 48:2179-2184 (2004); and P. J. Petersen et. al., Antimicrob. Agents Chemother. 43:738-744(1999).

In one embodiment, the pharmaceutical composition may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, the pharmaceutical composition may be used to treat or control bacterial infections in warm-blooded animals caused by bacteria having the TetM and TetK resistant determinants. Also, the pharmaceutical composition may be used to treat bone and joint infections, catheter-related Neutropenia, obstetrics and gynecological infections, or to treat other resistant pathogens, such as VRE, ESBL, enterics, rapid growing mycobacteria, and the like.

In one embodiment, the pharmaceutical composition may be used in the treatment of bacterial infection in the gastrointestinal tract, such as the lower gastrointestinal tract.

In one embodiment, the anaerobe is Clostridium difficile.

### EXAMPLES

### Example 1

In this Example, the dissolution behavior of enteric coated tigecycline granules in capsules was investigated in a solution of 0.1N HCl, then in phosphate buffer pH 6.8 at 37° C. These conditions mimic the gastric system (0.1N) and the lower intestinal tract (pH 6.8).

The formulation used is described in Example 3, below.

Gelatin capsules of enteric coated granules of 100 mg tigecycline were added to three separate vessels (Capsules 1, 2, and 3). The capsules were dissolved with a USP Apparatus 2 (paddles) at 100 rpm in 750 mL of 0.1N HCl at 37° C. The dissolution was allowed to occur for 2 h, followed by addition of 250 mL of 0.2M Na₃PO₄. The pH of this mixture was adjusted to 6.8. Table I below lists the dissolution data.

TABLE I
Percent release of gelatin capsules of enteric
coated 100 mg tigecycline granules

| Time (min) | Cap 1 | Cap 2 | Cap 3 |
|---|---|---|---|
| 0 | 0 | 0 | 0 |
| 30 | 11.14271 | 12.56791 | 11.28477 |
| 60 | 24.17531 | 25.30732 | 22.83157 |
| 90 | 30.8192 | 30.66811 | 29.8502 |
| 120 | 35.07275 | 35.47755 | 33.74161 |
| 125 | 39.30319 | 38.94879 | 37.98354 |
| 130 | 40.70022 | 40.81831 | 38.93004 |
| 135 | 42.28829 | 43.52615 | 41.04458 |
| 150 | 49.00615 | 47.11648 | 47.38426 |
| 180 | 52.64652 | 51.85096 | 51.09949 |
| 240 | 75.78954 | 70.31774 | 67.92135 |
| 300 | 79.53955 | 79.71117 | 81.44953 |

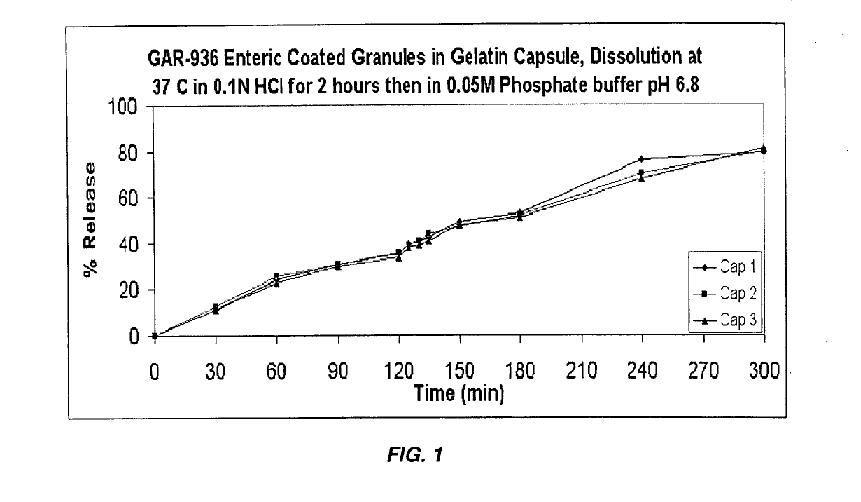

FIG. 1 is a plot of the data of Table I of percent release x-axis) versus time (min). The ratio of AUC to mg/ml is according to the equation y=16279x−58.773.

This Example demonstrates that the formulation releases substantially most of the tigecycline at higher pH, e.g., after 2 hours.

### Example 2

This Example demonstrates the oral bioavailability of tigecycline in cynomolgus monkeys when administered as an oral formulation (gavage). The pharmacokinetics of tigecycline after single oral and intravenous administration are also presented in this Example.

Male monkeys were first administered an oral (gavage) dose of 15 mg/kg of tigecycline and then an intravenous dose of 5 mg/kg of tigecycline after a one-week wash-out period.

Materials and Methods

Study Design

Four male cynomolgus monkeys were used in the study. In a first dosing period, each monkey was administered a single 15 mg/kg oral (gavage) dose of tigecycline in 0.9% saline. The dosing volume was 10 mL/kg. Blood samples (2 mL per sample) were obtained prior to dosing (0 hr) and at 0.5, 1, 2, 4, 6, 8, 12, 24, 32 and 48 hr after the oral dose. After a one-week washout period, each monkey was administered a single 5 mg/kg intravenous dose of tigecycline in 0.9% saline. Blood samples (2 mL) were obtained pre-dose (0 hr) and at 5 mm., 0.5, 1, 2, 4, 6, 8, 12, 24, 32 and 48 hr post-dose. Blood samples were collected using a stainless steel needle and vacutainer tube containing sodium heparin as the anticoagulant. Blood samples were placed on ice after collection and centrifuged at approximately 4° C. Plasma samples was separated, frozen and stored at approximately −70° C. prior to analysis.

Quantitation of Tigecycline in Monkey Plasma

Tigecycline concentrations were determined using an HPLC method that was previously validated in rat and dog plasma, although this method was modified to be used in monkey plasma. In this method, tigecycline in 0.2 mL of monkey plasma samples was extracted by protein precipitation with acetonitrile and the precipitated proteins were separated by centrifugation. The supernatant was evaporated and the extract was reconstituted in 0.05N HCl for HPLC analysis. Regression analysis was performed on the calibration curve using a quadratic fit with a weighting factor of 1/(concentration)². By using 0.2 mL of monkey plasma sample, the assay limit of quantitation (LOQ) was 100 ng/mL and the curve range was between 100 and 6400 ng/mL.

Pharmacokinetic Calculations

Pharmacokinetic parameters were calculated using the pharmacokinetics analysis program WinNonlin, version 2.1 (Scientific Consulting Inc.) from the individual animal concentration vs. time profiles. This program analyzes data using a model-independent approach and the standard methods described by Gibaldi and Perrier (Gibaldi M, Perrier D., Pharmacokinetics, 2^{nd} ed., Marcel Dekker, Inc., NY, 1982). For the purpose of this analysis, no attempt was made to back extrapolate the concentration immediately after the IV bolus dose, rather the concentration at 0 hr (C₀, immediately after dosing) was assumed to be equal to the first measured concentration (at 5 minutes, C₅ₘᵢₙ). To determine the mean plasma drug concentrations, all values below the lower limit of quantitation (LOQ=100 ng/mL) were treated as zero. The terminal half-life (t_{1/2}) was determined by 0.693/λ, where λ is the terminal rate constant and is determined by a log-linear fitting of the terminal portion of the concentration-time curve. AUC₀₋₄ was calculated by AUC₀₋ₜ+Cₜ/λ, where AUC₀₋ₜ was the AUC from time 0 to t, the last quantifiable time point and Cₜ was the last quantifiable concentration. The area under the plasma concentration-time curve from time 0 to t (AUC₀₋ₜ) was calculated using the linear trapezoidal method. Systemic clearance (CL_{T}) after the iv dose was calculated using the formula of Dose/AUC₀₋₄. The volume of distribution at steady-state (Vdₛₛ) was calculated using the formula of MRTᵢᵥ×CL_{T}, where MRTᵢᵥ is the mean residence time after iv dosing and equals AUMC₀₋₄/AUC₀₋₄. For the oral dose, Cₘₐₓ and tₘₐₓ values were obtained by inspection of the concentration vs. time curves. Due to the paucity of quantifiable concentrations after oral administration, the AUC₀₋₄ could not be calculated.

Analytical Performance of the HPLC Method for Tigecycline in Monkey Plasma

Five analytical runs were performed for the analysis of samples. The back-calculated values of the calibration curves are presented in Table II. The CV of tigecycline calibration standards were between 2.1 and 6.3% and the bias values ranged from −5.4 to 3.8%.

TABLE II
Analytical Performance of Tigecycline Assay in Monkey Plasma:
Back-Calculated Values of Tigecycline Calibration Standards

| | | Nominal concentration of tigecycline, ng/ML | | | | | | | | | |
|---|---|---|---|---|---|---|---|---|---|---|---|
| | | 100 | 200 | 400 | 500 | 800 | 1600 | 3200 | 4000 | 5000 | 6400 |
| | No. | Concentration of tigecycline found, ng/ML | | | | | | | | | |
| | 1 | 97.7 | 205 | 418 | 494 | 825 | 1604 | 3070 | 3861 | 4848 | 6709 |
| | 2 | 100 | 194 | 429 | NA | 763 | 1581 | 3284 | 3851 | 5158 | 6335 |
| | 3 | 100 | 202 | 416 | 478 | 724 | 1549 | 3510 | 4377 | 4829 | 6069 |
| | 4 | 103 | 189 | 404 | NA | 736 | 1652 | 3259 | 4300 | 5109 | 5996 |
| | 6 | 98.0 | 216 | 409 | 447 | 779 | 1512 | 3403 | 4297 | 5120 | 5968 |
| Mean | | 99.7 | 201 | 415 | 473 | 765 | 1580 | 3305 | 4137 | 5013 | 6215 |
| SD | | 2.12 | 10.4 | 9.52 | 23.9 | 39.8 | 53.3 | 165 | 259 | 160 | 312 |
| % CV | | 2.1 | 5.2 | 2.3 | 5.1 | 5.2 | 3.4 | 5.0 | 6.3 | 3.2 | 5.0 |
| % Bias | | −0.3 | 0.5 | 3.8 | −5.4 | −4.4 | −1.3 | 3.3 | 3.4 | 0.3 | −2.9 |
| n | | 5 | 5 | 5 | 3 | 5 | 5 | 5 | 5 | 5 | 5 |

NA: Not applicable

The calibration curve parameters are shown in Table III.

TABLE III
Analytical Performance of Tigecycline Assay in
Monkey Plasma: Calibration Curve Parameters

| | | 2^{nd} Order | 1^{st} Order | | |
|---|---|---|---|---|---|
| | Curve | Regression | Regression | | |
| | Number | Constant | Constant | Intercept | R² |
| | 1 | 0.0000 | 0.0000699 | −0.000908 | 0.9975 |
| | 2 | 0.0000 | 0.0000793 | −0.001800 | 0.9981 |
| | 3 | 0.0000 | 0.0000738 | −0.00262 | 0.9928 |
| | 4 | 0.0000 | 0.0000860 | −0.00348 | 0.9956 |
| | 6 | 0.0000 | 0.0000846 | −0.00274 | 0.9933 |
| Mean | | 0.0000 | 0.0000787 | −0.00231 | 0.9955 |
| SD | | 0.0000 | 0.0000069 | 0.000984 | 0.0024 |
| n | | 5 | 5 | 5 | 5 |

Regression analysis was performed with the following equation:
y=ax²+bx+c
where:
a=2^{nd} Order regression line constant.
b=1^{st} Order regression line constant.
C=Intercept.
y=Internal standard peak height ratio of tigecycline.
x=tigecycline concentration (ng/mL).

In all analytical runs, the coefficients of determination (R²) were >0.99. In all analytical runs, two replicates of low, mid-range and high QC samples were analyzed along with study samples. The low QC and the high QC have nominal concentrations of 300 and 3000 ng/mL, respectively. For the mid-range QC, the target nominal concentration was 900 ng/mL. Two separate batches of mid-range QC were prepared and both had concentrations below the target (ca. 600 ng/mL). The target concentrations of the mid-range QC batches were determined by analyzing four (batch A) or eight (batch B) replicates of each mid-range QC batch. Mid-range QC batch A (determined concentration of 663 ng/mL) was analyzed with curves 1 and 2. Mid-range QC batch B (determined concentration of 556 ng/mL) was analyzed with curves 3, 4 and 6. The results of QC samples from all analytical runs are shown in Table IV.

TABLE IV
Analytical Performance of Tigecycline Assay
in Monkey Plasma: Results of QC Samples

| | | Low | Mid A | Mid B | High |
|---|---|---|---|---|---|
| | Curve | (300 | (663 | (556 | (3000 |
| | Number | ng/mL) | ng/mL) | ng/mL) | ng/mL) |
| | 1 | 288 | 729 | NA | 3310 |
| | | 319 | 762 | NA | 3281 |
| | 2 | 294 | 664 | NA | 3273 |
| | | 276 | 699 | NA | 3037 |
| | 3 | 293 | NA | 538 | 3211 |
| | | 295 | NA | 578 | 3302 |
| | 4 | 280 | NA | 632 | 2743 |
| | | 252 | NA | 650 | 2828 |
| | 6 | 273 | NA | 535 | 2628 |
| | | 395 | NA | 610 | 2579 |
| Mean | | 297 | 714 | 591 | 3019 |
| SD | | 38.8 | 41.8 | 48.2 | 297 |
| % CV | | 13.1 | 5.9 | 8.2 | 9.8 |
| % Bias | | −1.0 | 7.7 | 6.3 | 0.6 |
| n | | 10 | 4 | 6 | 10 |

NA: Not applicable; this QC batch was not analyzed with this run.

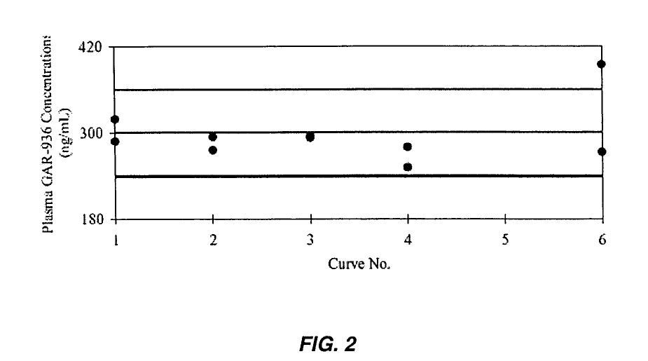

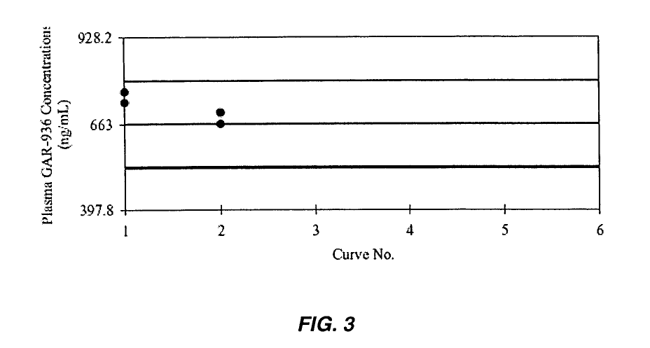

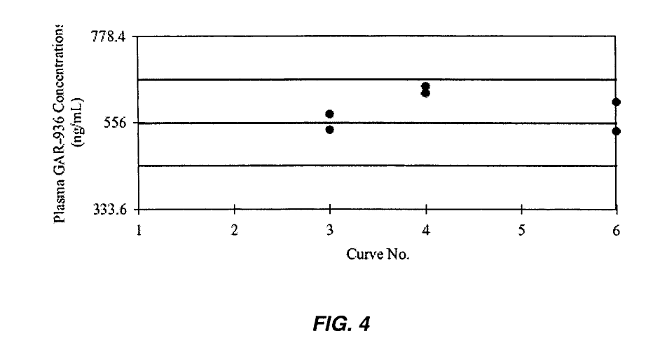

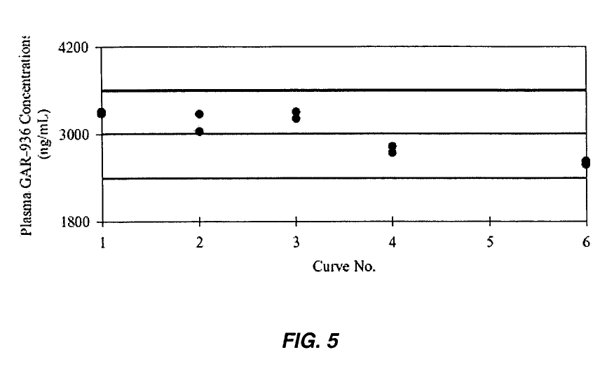

The CV of QC samples were between 5.9 and 13.1% and the biases were between −1.0 and 7.7%. The QC results are also depicted in QC charts and they are presented in FIGS. 2 to 5.

Pharmacokinetics of Tigecycline in Cynomolgus Monkeys

The concentrations of tigecycline after a single 15 mg/kg oral dose in monkeys are presented in Table V.

TABLE V
Plasma Concentrations (ng/mL) of Tigecycline in Monkeys
After a Single Oral (gavage) Dose of 15 mg/kg

| Animal | Hours | | | | | | | | | | |
|---|---|---|---|---|---|---|---|---|---|---|---|
| No. | 0 | 0.5 | 1 | 2 | 4 | 6 | 8 | 12 | 24 | 32 | 48 |
| 1 | <100 | <100 | 114 | 131 | <100 | <100 | <100 | <100 | <100 | <100 | <100 |
| 2 | <100 | 101 | 128 | 191 | <100 | <100 | <100 | <100 | <100 | <100 | <100 |
| 3 | <100 | 121 | 178 | <100 | <100 | <100 | <100 | <100 | <100 | <100 | <100 |
| 4 | <100 | <100 | 105 | 150 | <100 | <100 | <100 | <100 | <100 | <100 | <100 |
| Mean | 0 | 55.5 | 131 | 118 | 0 | 0 | 0 | 0 | 0 | 0 | 0 |
| SD | 0 | 64.6 | 32.6 | 82.6 | 0 | 0 | 0 | 0 | 0 | 0 | 0 |
| n | 4 | 4 | 4 | 4 | 4 | 4 | 4 | 4 | 4 | 4 | 4 |

The concentrations of tigecycline after a single 5 mg/kg iv dose are shown in Table VI.

TABLE VI
Plasma Concentrations (ng/mL) of Tigecycline in Monkeys
After a Single Intravenous Dose of 5 mg/kg

| Animal | Hours | | | | | | | | | | | |
|---|---|---|---|---|---|---|---|---|---|---|---|---|
| No. | 0 | 0.083 | 0.5 | 1 | 2 | 4 | 6 | 8 | 12 | 24 | 32 | 48 |
| 1 | <100 | 15096 | 2030 | 1449 | 1228 | 721 | 517 | 429 | 264 | 167 | <100 | <100 |
| 2 | <100 | 8136 | 1724 | 1449 | 1193 | 938 | 630 | 457 | 325 | 216 | 127 | 108 |
| 3 | <100 | 14002 | 1890 | 1056 | 909 | 539 | 419 | 308 | 200 | 110 | <100 | <100 |
| 4 | <100 | 23050 | 3340 | 1661 | 1013 | 588 | 431 | 372 | 265 | 155 | <100 | <100 |
| Mean | 0 | 15071 | 2246 | 1404 | 1086 | 697 | 499 | 392 | 264 | 162 | 31.8 | 27.0 |
| SD | 0 | 6135 | 740 | 252 | 151 | 178 | 97.5 | 66.0 | 51.0 | 43.6 | 63.5 | 54.0 |
| n | 4 | 4 | 4 | 4 | 4 | 4 | 4 | 4 | 4 | 4 | 4 | 4 |

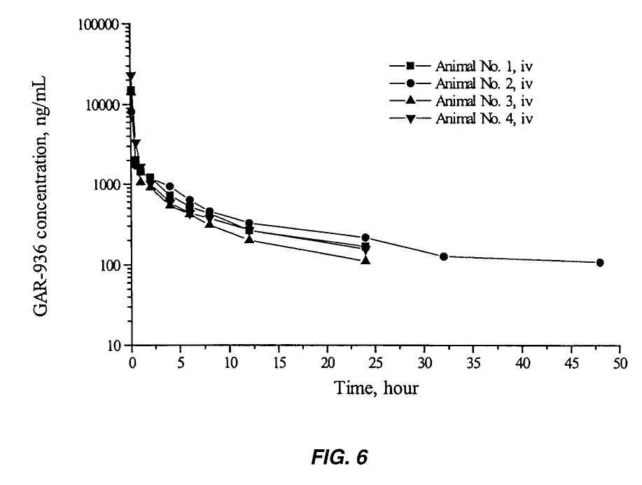

Plasma concentrations vs. time profiles after a single iv dose of tigecycline in monkeys are depicted in FIG. 6. Pharmacokinetic parameters from individual animals are tabulated in Table VII.

TABLE VII
Individual and Mean (±SD) Pharmacokinetic Parameters of Tigecycline in Monkeys After
a Single Oral (gavage) Dose of 15 mg/kg or After a Single Intravenous Dose of 5 mg/kg

| Dose | | Animal | Cₘₐₓ^{a} | tₘₐₓ | AUC₀₋ₜ | AUC0₋₄ | t_{1/2} | Cl_{T} | Vdₛₛ | MRTᵢᵥ |
|---|---|---|---|---|---|---|---|---|---|---|
| (mg/kg) | Route | No. | (ng/mL) | (hr) | (ng · hr/mL) | (ng · hr/mL) | (hr) | (L/kg/hr) | (L/kg) | (hr) |
| 15 | oral | 1 | 131 | 2.0 | 151^{b} | nc | nc | NA | NA | NA |
| | | 2 | 191 | 2.0 | 242^{b} | nc | nc | NA | NA | NA |
| | | 3 | 178 | 1.0 | 105^{c} | nc | nc | NA | NA | NA |
| | | 4 | 150 | 2.0 | 154^{b} | nc | nc | NA | NA | NA |
| | | Mean | 163 | 1.8 | 163 | — | — | — | — | — |
| | | SD | 27.1 | 0.5 | 57.2 | — | — | — | — | — |
| | | n | 4 | 4 | 4 | | | | | |
| 5 | iv | 1 | 15096 | NA | NA | 18220 | 12.8 | 0.274 | 3.13 | 11.4 |
| | | 2 | 8136 | NA | NA | 20662 | 19.1 | 0.242 | 5.02 | 20.7 |
| | | 3 | 14002 | NA | NA | 14007 | 11.4 | 0.357 | 3.28 | 9.1 |
| | | 4 | 23050 | NA | NA | 20178 | 13.2 | 0.248 | 2.45 | 9.8 |
| | | Mean | 15071 | — | — | 18267 | 14.1 | 0.280 | 3.47 | 12.8 |
| | | SD | 6135 | — | — | 3030 | 3.4 | 0.053 | 1.09 | 5.4 |
| | | n | 4 | | | 4 | 4 | 4 | 4 | 4 |

^{a}Cₘₐₓ = C_{5 min.} after the iv dose.
^{b}t = 2 hr for AUC determination.
^{c}t = 1 hr for AUC determination.
NA: Not applicable.
nc: AUCC0₋₄ or t_{1/2} value not calculated due to insufficient data in the apparent terminal phase.

After a single 15 mg/kg oral (gavage) dose, tigecycline was detected in samples up to 2 hours post-dose. The mean (±SD) Cₘₐₓ value was 163±27.1 ng/mL and the tₘₐₓ values were between 1 and 2 hours. Due to the paucity of quantifiable concentrations in the terminal phase of the concentration vs. time curves after oral dosing AUC₀₋₄, and t_{1/2} values were not estimated after the oral dose. Also, due to the limited number of time points with quantifiable tigecycline concentration and the partial AUC values estimated, absolute bioavailability of tigecycline after oral dosing could not be determined.

A 0.5% blood bioavailability is suitable for treating GI tract infections since the desired site of action is in the GI tract and not in the blood. Thus, a 0.5% blood bioavailability can translate to approximately 99% bioavailability in the GI tract.

After a single 5 mg/kg intravenous dose in monkeys, the plasma concentrations of tigecycline declined polyexponentially. The mean t_{1/2} value estimated from the terminal phase of the plasma concentration vs. time curves was 14.1±3.4 hours, that was similar to the MRTᵢᵥ of 12.8±5.4 hours. The mean (±SD) AUC₀₋₄, value of tigecycline was 18267±3030 ng·hr/mL. The mean tigecycline CI_{T} was 0.280±0.053 L/kg/hr and the mean Vdₛₛ was 3.47±1.09 L/kg.

Discussion

The results of this study showed that the blood bioavailability of tigecycline was low after oral administration. When treating GI tract infections, low blood bioavailability is desired because the drug is kept within the stomach for local action against the organisms in the GI tract. The absolute bioavailability could not be estimated after a single 15 mg/kg oral dose due to insufficient data in the terminal phase for the estimation of AUC₀₋₄ values. After a single iv dose in monkeys, the plasma concentrations of tigecycline declined polyexponentially. The terminal half-lives estimated from the terminal phase of the plasma concentration vs. time curves were between 11.4 and 19.1 (mean 14.1) hours and were similar to the MRTᵢᵥ (mean 12.8 hours). The systemic clearance (Cl_{T}) of GAR-93 6 in monkeys was relatively low (mean 0.280 L/kg/hr) but similar to that in dogs (ca. 0.26 L/kg/hr after a single 5 mg/kg dose). The steady-state volume of distribution (Vdₛₛ) of tigecycline in monkeys was large (3.47 L/kg) and in excess of the volume of total body water in this species (see Davies B, Morris T. “Physiological parameters in laboratory animals and humans,” Pharm. Res. 1993; 10:1093-95), suggesting that tigecycline should be distributed to various tissues and organs.

### Example 3

This Example demonstrates the oral bioavailability in fasted male cynomolgus monkeys from an encapsulated microparticulate (100 mg) formulation administered as a single enteric coated oral formulation. Tigecycline plasma concentrations were determined for the formulation type by an LC/MS/MS method.

Materials and Methods

Formulation

The tigecycline formulation was a 100 mg, encapsulated multi-particulate formulation having the components listed in Table VIII below:

TABLE VIII

| Granulation | % w/w | mg/250 mg |
|---|---|---|
| Tigecycline, 98% potency | 30.00 | 76.53 |
| Microcrystalline cellulose (Avicel PH101)^{a} | 22.00 | 53.47 |
| Mannitol DC grade | 30.00 | 75.00 |
| HPMC K100 (Dow) | 5.00 | 12.50 |
| Sodium Phosphate (dibasic) | 8.00 | 20.00 |
| Sodium stearyl fumarate (Pruv) | 1.50 | 3.75 |
| EDTA | 0.50 | 1.25 |
| Sodium starch glycolate | 3.00 | 7.50 |

^{a}Potency of tigecycline is adjusted against microcrystalline cellulose (MCC)

The enteric coating comprised a Seal Coat, YS-1-7006, and Enteric Coat (Acryl-EZE). The final potency for enteric coated tigecycline was 209 mg/g. Each 100 mg capsule contained 478.5 mg enteric coated granules.

Experimental Design and Sample Collection

The bioavailability of tigecycline was investigated with four male cynomolgus monkeys, each having body weights ranging from 5.5 to 7.1 kg. The monkeys were housed in Bioresources vivarium with free access to water and food. The four monkeys received the oral formulation described above (1×100 mg multi-particulate capsule). The formulation was administered with 10 mL water. All monkeys were fasted overnight prior to dosing (with free access to water) and were fed 4 hours after dose administration.

Blood samples were drawn from the saphenous vein at 0 (predose), 0.5, 1, 2, 3, 4, 8, 12 and 24 hours after dosing. Approximately 3 mL of blood were drawn into Vacutainer® tubes containing sodium heparin as the anticoagulant. Plasma was separated in a refrigerated centrifuge and stored at −70° C. Plasma samples were delivered to the assay site packed on dry ice.

Plasma tigecycline concentrations were determined by an LC/MS/MS method described above. Based on a 0.5 mL sample volume, the method has a limit of quantitation of 10 ng/mL.

Determination of Tigecycline Concentrations in Monkey Plasma

Tigecycline concentrations were determined by an LC/MS/MS method. Using 0.50 mL of sodium heparin monkey plasma, the lower limit of quantitation (LLOQ) was 10.0 ng/mL and the assay range was 10.0 to 1000 ng/mL. To monitor assay performance, all analytical runs were analyzed with low, mid-range, and high concentration (30, 300, and 800 ng/mL nominal concentrations) quality control samples (QCs) in quintuplets.

Analytical Performance of Tigecycline LC/MS/MS Assay in Monkey Plasma

There was one analytical run for the quantitation of tigecycline in monkey plasma samples from this study. The back-calculated values of tigecycline calibration standards prepared in monkey plasma and the calibration curve regression constants are shown in Table IX.

TABLE IX
Analytical Performance of Tigecycline Assay in Monkey
Plasma: Back-Calculated Concentrations of Calibration
Standards and Calibration Curve Regression Constants
(A) Back-Calculated Concentrations of Tigecycline Calibration
Standards in Monkey Plasma

| Curve | Tigecycline Nominal Concentration, ng/mL | | | | | | | |
|---|---|---|---|---|---|---|---|---|
| No. | 10 | 25 | 50 | 100 | 200 | 400 | 900 | 1000 |
| | Tigecycline Observed Concentration, ng/mL | | | | | | | |
| 1 | 9.72 | 25.3 | 51.9 | 113 | 221 | 384 | 796 | 895 |
| Mean | 9.72 | 25.3 | 51.9 | 113 | 221 | 384 | 796 | 895 |
| % Bias | −2.8 | 1.2 | 3.8 | 13.0 | 10.5 | −4.0 | −11.6 | −10.5 |
| n | 1 | 1 | 1 | 1 | 1 | 1 | 1 | 1 |

(B) Calibration Curve^{a} Regression Constants for Tigecycline
Assay in Monkey Plasma

| | Curve No. | Slope | Intercept | R² |
|---|---|---|---|---|
| | 1 | 0.00190 | 0.00917 | 0.9895 |
| Mean | | 0.00190 | 0.00917 | 0.9895 |
| n | | 1 | 1 | 1 |

^{a}A linear regression method was used with 1/concentration² as the weighting factor.

Linear regression was performed using a weighting factor of 1/(concentration)². The mean biases of back-calculated calibration standards ranged from −11.6% to 13.0%. The R² value of the calibration curve was 0.9895.

Results of tigecycline quality control (QC) samples prepared in monkey plasma and analyzed with the study samples are summarized in Table X.

TABLE X
Analytical Performance of Tigecycline Assay in Monkey
Plasma: Results of Quality Control (QC) Samples
Tigecycline QC Samples

| | Curve | Low QC | Middle QC | High QC |
|---|---|---|---|---|
| | Number | (30 ng/mL) | (300 ng/mL) | (800 ng/mL) |
| | 1 | 28.1 | 279 | 702 |
| | | 27.3 | 277 | 682 |
| | | 28.6 | 261 | 690 |
| | | 30.1 | 302 | 666 |
| | | 31.8 | 296 | 691 |
| Mean | | 29.2 | 283 | 686 |
| S.D. | | 1.79 | 16.3 | 13.3 |
| % CV | | 6.1 | 5.8 | 1.9 |
| % Bias | | −2.7 | −5.7 | −14.3 |
| n | | 5 | 5 | 5 |

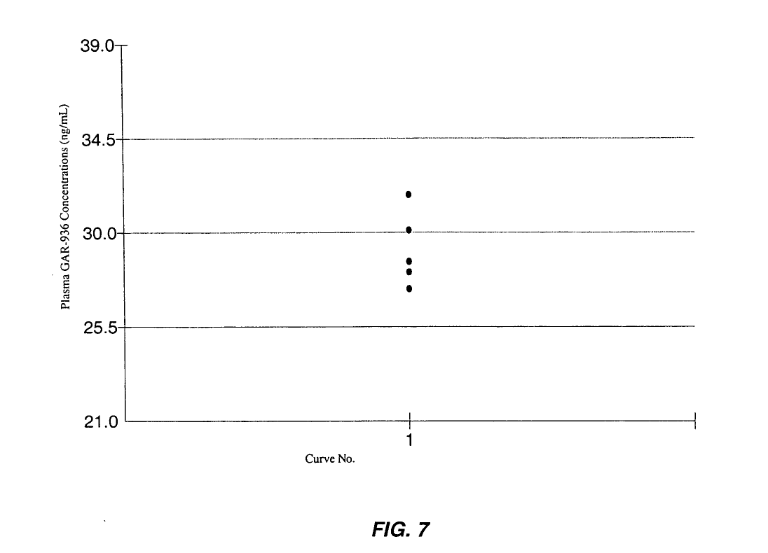

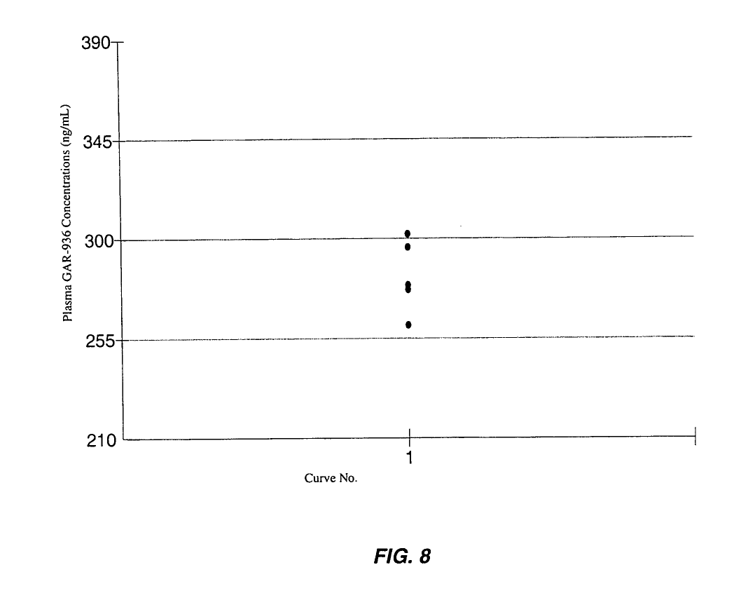

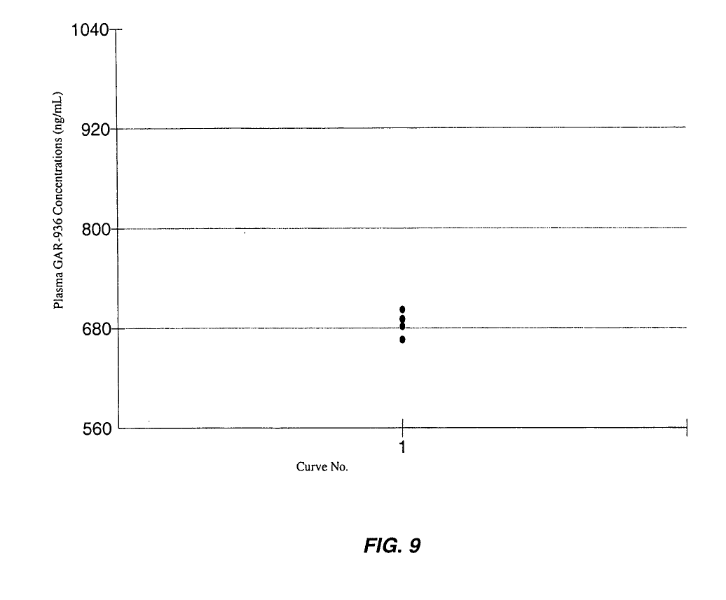

The CV of the QC samples ranged from 1.9% to 6.1% and the mean biases ranged from −14.3% to −2.7%. The QC results are also depicted graphically in FIGS. 7 to 9.

Plasma Concentrations of Tigecycline in Monkeys

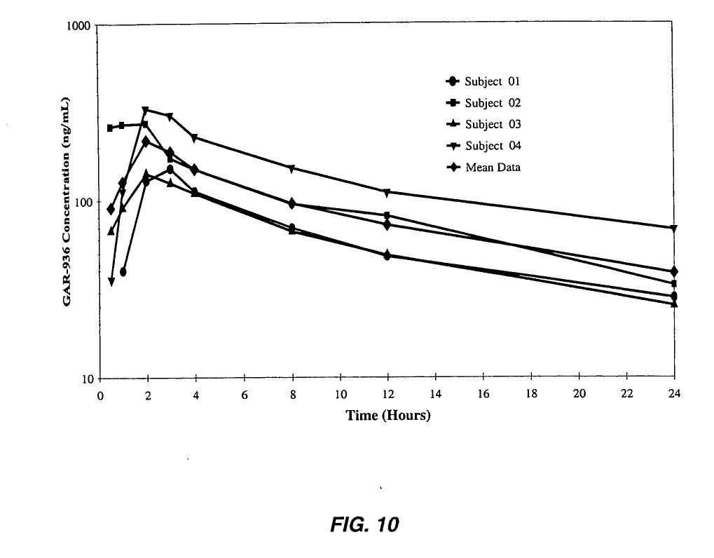

Tigecycline plasma concentrations (ng/mL) in fasted monkeys after a single oral dose (100 mg capsule) of tigecycline from an encapsulated microparticulate formulation are presented in Table XI and shown graphically in FIG. 10.

TABLE XI
Plasma Concentrations (ng/mL) of Tigecycline After A Single
Oral Dose (100 mg Tigecycline Encapsulated Microparticulate
Capsule) in Fasted Male Cynomolgus Monkeys

| | 0 hr | 0.5 hr | 1 hr | 2 hr | 3 hr | 4 hr | 8 hr | 12 hr | 24 hr |
|---|---|---|---|---|---|---|---|---|---|
| SAN* | Tigecycline Concentration, ng/mL | | | | | | | | |
| 1 | <10.0 | <10.0 | 39.9 | 130 | 152 | 113 | 69.6 | 48.1 | 28.1 |
| 2 | <10.0 | 261 | 270 | 273 | 174 | 151 | 95.3 | 81.6 | 33.1 |
| 3 | <10.0 | 67.4 | 90.9 | 143 | 126 | 110 | 66.6 | 48.8 | 25.4 |
| 4 | <10.0 | 35.6 | 113 | 331 | 304 | 230 | 153 | 111 | 68.2 |
| Mean | 0 | 91.0 | 128 | 219 | 189 | 151 | 96.1 | 72.4 | 38.7 |
| SD | 0 | 117 | 99.2 | 98.6 | 79.1 | 55.9 | 40.0 | 30.1 | 19.9 |
| % CV | 0 | 128.6 | 77.5 | 45.0 | 41.9 | 37.0 | 41.6 | 41.6 | 51.4 |
| n | 4 | 4 | 4 | 4 | 4 | 4 | 4 | 4 | 4 |

*SAN: Study animal number

Plasma Concentration-Time Data Analysis

Noncompartmental analysis of the individual monkey plasma tigecycline concentration-time profiles was performed using WinNonlin, Model 200. Area under the plasma tigecycline concentration-time curves (AUC) were calculated by log/linear trapezoid rule. The peak plasma tigecycline concentrations (Cₘₐₓ) and the time to reach Cₘₐₓ (tₘₐₓ) were noted directly from the plasma tigecycline concentration-time profiles.

The AUC (ng·hr/mL, mean ±SD) value for the formulation was 2830±1111. The Cₘₐₓ value (ng/mL, mean ±SD) for the formulation was 225±92.4.

Pharmacokinetics

The individual and mean monkey pharmacokinetic parameters are reported in Table XII.

TABLE XII
Individual and Mean Pharmacokinetic Parameters of Tigecycline
After A Single Dose (100 mg Encapsulated Microparticulate Capsule.
Batch L23290-29B) in Fasted Male Cynomolgus Monkeys

| Monkey | Dose | Cmax | Tmax | AUC₀₋₂₄ | AUC_{0-∞} | T1/2 | | |
|---|---|---|---|---|---|---|---|---|
| SAN | (mg/kg) | (ng/mL) | (hr) | (ng hr/mL) | ₍ng hr/mL) | (hr) | AUC/Dose | Cmax/Dose |
| 01 | 14.1 | 152 | 3.0 | 1430 | 1950 | 12.8 | 138 | 10.8 |
| 02 | 14.9 | 273 | 2.0 | 2390 | 2840 | 9.48 | 191 | 18.3 |
| 03 | 16.7 | 143 | 2.0 | 1460 | 1890 | 11.8 | 113 | 8.56 |
| 04 | 18.2 | 331 | 2.0 | 3220 | 4640 | 14.4 | 255 | 18.2 |
| Mean | 16.0 | 225 | 2.25 | 2130 | 2830 | 12.1 | 174 | 14.0 |
| S.D. | 1.83 | 92.4 | 0.5 | 855 | 1111 | 2.06 | 62.7 | 5.04 |
| % CV | 11.4 | 41.1 | 22.2 | 40.2 | 39.2 | 17 | 36.0 | 36.1 |
| n | 4 | 4 | 4 | 4 | 4 | 4 | 4 | 4 |

Table XIII compares the mean pharmacokinetic parameters and the absolute and relative bioavailability of tigecycline in the encapsulated multi-particulate formulation to the 0.9% saline tigecycline solution administered IV and orally (gavage), as described in Example 2 above.

TABLE XIII
Comparison of Pharmacokinetic Parameters [Mean
(n = 4)] in Male Cynomolgus Monkeys After
A Single Dose Administration of Tigecycline

| | | 15 mg/kg | |
|---|---|---|---|
| | 16 mg/kg 100 | 0.9% saline, | 15 mg/kg |
| Parameter | mg oral capsule | Gavage¹ | IV Gavage |
| AUC_{0-t or 0-∞} | 2830 | 163 | 18267 |
| AUC/Dose | 174 | 10.9 | 3653 |
| Cmax (ng/mL) | 225 | 163 | 15071 |
| Cmax/Dose | 14.0 | 10.9 | 3014 |
| tmax (hr) | 2.25 | 1.8 | Not applicable |
| t½ (hr) | 12.1 | Not calculated | 14.1 |
| Bioavailability | 4.8% | — | — |

See Example 2

The AUC (ng·hr/mL, mean ±SD) value for the formulation was 2830±1111. The Cₘₐₓ values (ng/mL, mean ±SD) for the formulation was 225±92.4.

A bioavailability study of a tigecycline formulation has been conducted in cynomolgus monkeys to assess the bioavailability of an enhanced encapsulated microparticulate oral dosage formulation.

The results of this study showed that the absolute bioavailability of tigecycline in the blood was 5% after oral administration. The capsule formulation (16 mg/kg) demonstrated significantly higher oral exposure (AUC) values as compared to previous studies conducted by preclinical development at 15 mg/kg.

When treating bacterial infections, a blood bioavailability of at least 5% can be suitable. For treating GI tract infections, a 5% blood bioavailability can translate to 95% availability in the GI tract.

### Example 4

This Example describes a dry powder layering process for the preparation of an oral formulation. Table XIV lists the formulation ingredients.

TABLE XIV

| Ingredient | % w/w | mg/250 mg |
|---|---|---|
| Tigecycline (98% | 60.0 | 150.00 |
| potency) | | |
| lactose | 31.5 | 78.75 |
| Sodium phosphate | 5.0 | 12.5 |
| (dibasic) | | |
| EDTA | 0.5 | 1.25 |
| Hypromellose solution | 5-10% solution | |
| Enteric Coat (Acryl- | 10-30% weight | |
| EZE), 93018429 | gain on dry | |
| | layered pellets | |

In this example the tigecycline, lactose, sodium phosphate and EDTA were blended together and fed through a screw feed into a fluid bed rotor granulator containing sucrose or microcrystalline spheroids. A 5-10% binder solution of hypromellose was sprayed simultaneously into the spinning bed of spheroids while the tigecycline blend was slowly added. After the desired quantity of tigecycline blend was added to the spheres, they were dried and discharged for enteric coating. Enteric coating was applied via a fluid bed processor using polymethacrylates. Other enteric polymers normally used in industry can also be used.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

1. A pharmaceutical composition comprising tigecycline having at least one enteric coating.

2. The composition according to claim 1, wherein the at least one enteric coating is chosen from dimethylaminoethyl methacrylatemethylacrylate acid ester copolymer, anionic acrylic resins such as methacrylic acid/methyl acrylate copolymer and methacrylic acid/ethyl acrylate copolymer, ethylacrylate-methylmethacrylate copolymer, hydroxypropylmethylcellulose acetate succinate (HPMCAS), hydroxypropylmethylcellulose phthalate (HPMCP), cellulose acetate phthalate (CAP), carboxymethylcellulose acetate phthalate (CMCAP), hydroxypropylmethylcellulose, hydroxyethylcellulose, methylhydroxyethylcellulose, sodium carboxymethylcellulose, hydroxypropylcellulose, polyvinyl pyrrolidone, shellac, methylcellulose, and ethylcellulose, and blends and copolymers thereof.

3. The composition according to claim 1, wherein the composition is in oral dosage form.

4. The composition according to claim 3, wherein the oral dosage form is chosen from capsules, tablets, pills, powders, granules, and lyophilized cakes and powders.

5. The composition according to claim 1, wherein the tigecycline is multi-particulate.

6. The composition according to claim 5, wherein the multi-particulate tigecycline has a mean particle size ranging from 0.3 mm to 1.5 mm.

7. The composition according to claim 1, further comprising at least one base.

8. The composition according to claim 7, wherein the at least one base is chosen from phosphates, carbonates, bicarbonates, citrates, and tartrates.

9. The composition according to claim 8, wherein the at least one base is chosen from sodium phosphates, sodium carbonate, sodium bicarbonate, and sodium citrate.

10. The composition according to claim 1, further comprising at least one chelating agent.

11. The composition according to claim 10, wherein the at least one chelating agent is chosen from EDTA EGTA, citrates, and tartrates.

12. The composition according to claim 1, further comprising at least one biopolymer.

13. The composition according to claim 12, wherein the at least one biopolymer is chosen from hypromellose, xanthan gum, and carbomer.

14. The composition according to claim 1, further comprising at least one base, at least one chelating agent, and at least one biopolymer.

15. The pharmaceutical composition of claim 5, comprising enteric coated multi-particulate pellets incorporated into a hard gelatin capsule, each pellet comprising tigecycline and microcrystalline cellulose, and at least one component chosen from at least one base, at least one chelating agent, and at least one biopolymer.

16. The pharmaceutical composition of claim 4, comprising an enteric coated tablet comprising tigecycline and microcrystalline cellulose, and further comprising at least one component chosen from at least one base, at least one chelating agent, and at least one biopolymer.

17. The pharmaceutical composition of claim 5, comprising multi-particulate pellets incorporated into an enteric coated soft gelatin capsule, each pellet comprising tigecycline and microcrystalline cellulose, and further comprising at least one component chosen from at least one base, at least one chelating agent, and at least one biopolymer.

18. A pharmaceutical composition comprising an enteric coated soft liquid gel capsule, and further comprising a non-aqueous solution of tigecycline and at least one component chosen from at least one base, at least one chelating agent, and at least one biopolymer.

19. A method of preparing a pharmaceutical composition comprising coating tigecycline with at least one enteric coating.

20. A method of treating at least one bacterial infection, comprising:
orally administering to a subject in need thereof a pharmaceutical composition comprising a therapeutically effective amount of tigecycline having at least one enteric coating.

21. The method according to claim 20, wherein the at least one bacterial infection is chosen from complicated intra-abdominal infections (cIAI), complicated skin and skin structure infections (cSSSI), Community Acquired Pneumonia (CAP), Hospital Acquired Pneumonia (HAP) indications, bacterial infections caused by bacteria having the TetM and TetK resistant determinants, bone and joint infections, catheter-related Neutropenia, obstetrics and gynecological infections, and bacterial infections caused by VRE, ESBL, enterics, and rapid growing mycobacteria.

22. The method according to claim 20, wherein the tigecycline is multi-particulate tigecycline.

23. A method of treating antibiotic associated pseudomembranous colitis caused by C. difficile and enterocolitis caused by S. aureus and associated methicillin resistant strains comprising:
orally administering to a subject in need thereof a pharmaceutical composition comprising a therapeutically effective amount of tigecycline having at least one enteric coating.

24-27. (canceled)
